SEQUENCE LISTING (12 sequences)

SEQ ID NO: 1 - protein, 8 aa, Artificial Sequence
  CLP313
MWVPVVFL

SEQ ID NO: 2 - protein, 9 aa, Artificial Sequence
  CLP314
VLVHPQWVL

SEQ ID NO: 3 - protein, 9 aa, Artificial Sequence
  CLP316
KLQCVDLHV

SEQ ID NO: 4 - protein, 9 aa, Artificial Sequence
  CLP315
DLPTQEPAL

SEQ ID NO: 5 - protein, 9 aa, Artificial Sequence
  CLP317
DLHVISNDV

SEQ ID NO: 6 - protein, 9 aa, Artificial Sequence
  CLP320
PLYDMSLLK

SEQ ID NO: 7 - DNA, 24 bp, Artificial Sequence
  CLP313
atgtgggtcccggttgtcttcctc

SEQ ID NO: 8 - DNA, 27 bp, Artificial Sequence
  CLP314
gttctggtgcacccccagtgggtcctc

SEQ ID NO: 9 - DNA, 27 bp, Artificial Sequence
  CLP316
aaacttcagtgtgtggacctccatgtt

SEQ ID NO: 10 - DNA, 27 bp, Artificial Sequence
  CLP315
gacctgcccacccaggagccagcactg

SEQ ID NO: 11 - DNA, 27 bp, Artificial Sequence
  CLP317
gacctccatgttatttccaatgacgtg

SEQ ID NO: 12 - DNA, 27 bp, Artificial Sequence
  CLP320
ccgctctacgatatgagcctcctgaag

(19) United States
(12) Patent Application Publication
(10) Pub. No.: US 2002/0132976 A1
(43) Pub. Date: Sep. 19, 2002

(54) Immunogenic peptides derived from prostate-specific antigen (PSA) and uses thereof

(76) Inventors: Artur Pedyczak, Pickering (CA); Pele Chong, Richmond Hill (CA); Charles Dwo Yuan Sia, Toronto (CA)

Correspondence Address: Micheline Gravelle, Bereskin & Parr, Box 401, 40 King Street West, Toronto, M5H 3Y2, CA

(21) Appl. No.: 09/829,004

(22) Filed: Apr. 10, 2001

Related U.S. Application Data
(60) Provisional application No. 60/195,456, filed on Apr. 10, 2000.

(51) Int. Cl. C07H 21/04; C12P 21/04; C12N 5/06; C07K 7/06; C07K 14/435

(52) U.S. Cl. 530/328; 530/350; 435/69.7; 536/23.5; 435/320.1; 435/325

(57) ABSTRACT

The identification of immunogenic peptides of PSA, nucleic acids coding therefor, and recombinant nucleic acids into which are inserted said nucleic acids coding for PSA peptides are disclosed. These peptides, nucleic acids and recombinant nucleic acids may be used in isolation, or as compositions thereof to modulate immune responses in animals. The invention further encompasses methods per se of modulating immune responses in animals.

The application claims benefit from U.S. Provisional Application Serial No. 60/195,456 filed Apr. 10, 2000 which is incorporated herein by reference in its entirety.

### FIELD OF THE INVENTION

The present invention relates to immunology, in particular to peptides derived from prostate-specific antigen (PSA) and nucleic acids coding therefor, recombinant nucleic acids into which are inserted nucleic acids coding for PSA peptides, and their use as immunogenic agents.

### BACKGROUND OF THE INVENTION

Cancer of the prostate is the most commonly diagnosed cancer in man and is the second most common cause of cancer death (Carter, H. B. and Coffey, D. S. (1990) Prostate 16:39-48; Armbruster, D. A. (1993) Clin Chem 39:181-195). If detected at an early stage, prostate cancer is potentially curable. However, a majority of cases are diagnosed at later stages when metastasis of the primary tumor has already occurred (Wang, M. C., Kuriyama, M., Papsidero, L. D., Loor, R. M., Valenzuela, L. A., Murphy, G. P., and Chu, T. M. (1982) Methods in Cancer Research 19:179-197). Present treatments for prostate cancer include radical prostatectomy, radiation therapy, or hormonal therapy. No systemic therapy has clearly improved survival in cases of hormone refractory disease. With surgical intervention, complete eradication of the tumor is not always achieved and the observed reoccurrence of the cancer (12-68%) is dependent upon the initial clinical tumor stage (Zietman, A. L., Shipley, W. L., and Willett, C. G. (1993) Cancer 71:959-969). Thus, alternative methods of treatment including prophylaxis or prevention are desirable.

The advent of DNA technology and its expanding use in the field of immunology has led to the identification of human tumor-associated antigens (TAAs) (Rosenberg, S. A. (1995) Cancer J. Sci. Am. 12:89-100; Boon, T. (1993) Int. J. Cancer 54:177-180). These TAAs now offer the potential of applying modern immunotherapeutic approaches to the treatment of some human cancers. The basis of one such approach is to immunize a host with appropriate immunogen(s) to elicit cell-mediated immunological responses involving recruitment of tumor-specific effector cells (e.g. cytotoxic T lymphocytes (CTLs)) in an effort to recognize and destroy neoplastic cells.

The human TAAs that have been identified to date can be classified into four general categories. The first encompasses the ‘cancer/testis’ antigens, such as those of the MAGE gene family, whose expression is tumor-specific. The second encompasses antigens which are virally-derived, such as those from human papilloma virus (HPV) and Epstein-Barr virus (EBV). The third encompasses differentiation antigens, including the prostate-specific antigen (PSA), prostate-specific membrane antigen (PSMA), Melan-A/MART-1, tyrosinase, gp100 and ganglioside GM2. The fourth encompasses antigens such as modified/mutated β-catenin, ras and p53, which are not normally found in a modified/mutated form in normal cells.

Within the specific context of prostate cancer, several potentially suitable TAAs have been identified. The first widely studied antigen demonstrated to be over-represented in the prostate gland in cancer modalities was prostatic acid phosphatase (PAP). Arguably, elevated levels of PAP in the bloodstream are considered to be indicative of prostate cancer (Yam (1974) Amer J Med 56:604). Improved methods of cancer detection using this enzyme have been described (WO 79/00475). The biochemical/biophysical structure of the enzyme has also been well studied (Sharief, F. S., et al., (1992) Biochem Biophys Res Commun 184:1468-1476; Van Etten, R. L., et al., (1991) J Biol Chem 266:9993-9999). Additionally, the nucleotide sequence encoding human PAP has been determined (Sharief, F. S., et al., (1989) Biochem Biophys Res Commun 180:79-86; Tailor, P. G., et al., (1990) Nucleic Acid Res 18:4928).

In addition to PAP, other antigen candidates over-represented in prostatic cancer tissue have been identified and/or characterized. The most prominent among these is human prostate specific antigen (PSA). PSA is a member of the glandular kallikrein family and is a protease with a restricted chymotrypsin-like specificity and is present in the epithelial cells comprising the prostatic ductal elements. It has been demonstrated in normal prostate and in all primary and metastatic tumor tissue tested, but not in nonprostatic cancer tissues or in normal tissues other than prostate.

The complete amino acid sequence of PSA from human seminal plasma has been determined (Watt K. W. et al. (1986) Proc Natl Acad Sci USA 83:3166-3170). PSA consists of a single polypeptide chain with 240 amino acid residues. There is evidence to suggest that the protein is glycosylated. A cDNA encoding PSA has also been isolated (Lundwall, A. and Lilja, H. (1987) FEBS Lett 214:317-322; Schultz, P. et al. (1988) Nucleic Acid Res 16:6226; Henttu, P. and Bihko, P. (1989) Biochem and Biophys Res Commun 160:903-910). The cellular gene for the PSA has also been characterized (Lundwall, A. (1989) Biochem and Biophys Res Commun 162:1151-1159, Riegman, P. H. J., et al. (1989) Biochem and Biophys Res Commun 159:103-111; Klobeck, G. et al. (1989) Nucleic Acid Res 17:3981).

A third prostate-related TAA of potential significance is prostate specific membrane antigen (PSMA). PSMA is similarly found in both benign and neoplastic prostate cells, albeit demonstrating a much greater presence in malignant cells by comparison to benign cells. The presence of PSMA has also been demonstrated on metastatic prostate cells. However, unlike PSA, PSMA is an integral membrane protein. cDNA encoding PSMA has been isolated and characterized (Israeli, R.S. et al. (1993) Cancer Res 53:227-230). The cDNA is 2.65 kilobases in length and portions thereof possess significant homology to the nucleic acid sequence coding for human transferrin receptor.

More recently, a number of reports have been published examining the potential usefulness of the prostate-related TAAs in the development of effective cancer immunotherapies. As is well known and documented, T-cell receptors on CD8⁺ T cells recognize a complex consisting of peptide (derived from antigen), β-2 microglobulin and class I major histocompatibility complex (MHC) heavy chain (i.e. HLA-A, B, or C in humans). As such, the identification of appropriate MHC class 1-restricted peptides (potentially derived from TAAs) might play an important role in the assessment and/or optimization of immunotherapeutic treatment strategies. Two important factors must be considered in the assessment of the potential of any TAA epitopes. These are: (1) the peptide must demonstrate an ability to lodge into the peptide binding groove of the respective class 1 MHC molecule and, (2) the peptide must possess sufficiently high binding affinity for the class 1 MHC molecules such that the peptide/class 1 MHC/B2-microglobulin (B2-M)-trimolecular complex displayed on antigen presenting cells is sufficiently stable for presentation, thus resulting in the subsequent activation of the appropriate subset of CD8⁺ effector cell.

In view of the foregoing, there is a need in the art to identify and characterize immunogenic peptides derived from PSA (and nucleic acids coding therefor) that can be used to develop effective immunotherapies.

### SUMMARY OF THE INVENTION

The inventors have identified a number of immunogenic peptides derived from prostate-specific antigen (PSA). The PSA derived peptides are useful in treating prostate cancer.

Accordingly, in one embodiment the present invention provides a PSA derived peptide comprising a sequence of the Formula I:

Xₙ—X₁—X—X—X—X—X—X—X₂

wherein

n=0 or 1;

each X₁ is independently selected from leucine or methionine;

each X₂ is independently selected from valine or leucine; and

each X is independently selected from any amino acid,

and fragments, elongations, analogs or derivatives of the PSA derived peptide.

In a preferred embodiment of the invention, the peptides are selected from the group consisting of MWVPVVFL (SEQ ID NO: 1), VLVHPQWVL (SEQ ID NO: 2), and KLQCVDLHV (SEQ ID NO: 3), or a fragment, analog, derivative or elongation of the PSA derived peptide.

In a further aspect, the present invention encompasses nucleic acids coding for the PSA derived peptides. In preferred embodiments, the nucleic acids have a sequence selected from the group consisting of the sequence of SEQ ID NO:7, SEQ ID NO:8, and SEQ ID NO:9. In yet further embodiments, the invention includes recombinant nucleic acids into which has been inserted a nucleic acid coding for a PSA peptide of the invention.

The invention also encompasses compositions. Accordingly, the invention includes compositions of PSA peptides, nucleic acids coding therefore, and/or recombinant nucleic acids into which has been inserted a nucleic acid coding for a PSA peptide of the invention. Said compositions may also comprise suitable adjuvants.

Methods of eliciting an immune response in a human are also encompassed by aspects of the invention. Accordingly, methods of eliciting an immune response in an animal comprising the administration of an effective amount of a PSA peptide, a nucleic acid coding therefor, and/or a recombinant nucleic acid into which has been inserted a nucleic acid coding for PSA peptide, and compositions thereof (with/without an adjuvant) are included within the scope of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from said detailed description.

### BRIEF DESCRIPTION OF THE FIGURES

FIG. 1 is a graphical representation of effector immune responses elicited in transgenic mice by a PSA peptide.

### DETAILED DESCRIPTION OF THE INVENTION

The following standard one letter and three letter abbreviations for the amino acid residues may be used throughout the specification: A, Ala—alanine; R, Arg—Arginine; N, Asn—Asparagine; D, Asp—Aspartic acid; C, Cys—Cysteine; Q, Gln—Glutamine; E, Glu—Glutamic acid; G, Gly—Glycine; H, His—Histidine; I, IIe—Isoleucine; L, Leu—Leucine; K, Lys—Lysine; M, Met—Methionine; F, Phe—Phenyalanine; P, Pro—Proline; S, Ser—Serine; T, Thr—Threonine; W, Trp—Tryptophan; Y, Tyr—Tyrosine; and V, Val—Valine.

I. PSA Derived Peptides

As hereinbefore mentioned, the present inventors have identified and characterized novel peptides, and nucleic acids encoding these novel peptides, which are derived from prostate-specific antigen (PSA).

Accordingly, the invention provides isolated PSA derived peptides and/or nucleic acids coding therefor which are capable of eliciting an immune response in an animal.

In one embodiment, the present invention provides a PSA derived peptide that is capable of eliciting an immune response comprising a sequence of the Formula I:

Xₙ—X₁—X—X—X—X—X₂

wherein

n=0 or 1;

each X₁ is independently selected from leucine or methionine;

each X₂ is independently selected from valine or leucine; and

each X is independently selected from any amino acid;

and fragments, elongations, analogs or derivatives of the PSA derived peptide.

The term “amino acid” includes all of the naturally occurring amino acids as well as modified amino acids.

Preferred peptides of Formula I include MWPVVFL (SEQ ID NO: 1), VLVHPQWVL (SEQ ID NO: 2), and KLQCVDLHV (SEQ ID NO: 3).

The phrases “PSA derived peptide(s)” and “PSA peptide(s)” as used herein mean a peptide of Formula I as described above and includes all analogs, derivatives, fragments and elongations thereof which maintain the ability to elicit an immune response in an animal. Preferably, the PSA derived peptide consists essentially of the sequence of the Formula I, more preferably, the PSA peptides are as shown in SEQ ID NOS:1-3. Collectively, the PSA derived peptides defined herein are referred to as the PSA peptides of the invention.

The term “analog” includes any peptide having an amino acid residue sequence substantially identical to the sequence of the PSA derived peptides shown herein in which one or more residues have been conservatively substituted with a functionally similar residue and which displays the ability to mimic a PSA derived peptide. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between threonine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that such polypeptide displays the requisite activity.

The term “derivative” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. PSA derived peptides of the present invention also include any peptide having one or more additions and/or deletions or residues relative to the sequence of a polypeptide whose sequence is shown herein, so long as the requisite activity is maintained or increased.

The term “fragment” refers to any subject peptide having an amino acid residue sequence shorter than that of a PSA peptide of the invention. Preferably, the fragment does not contain less than 5, more preferably not less than 8, amino acids.

The term “elongation” refers to any subject peptide having additional amino acid residues added to either end of the peptide, preferably from 1 to 10 amino acid residues, added to either the amino-terminal and/or carboxy-terminal end of a PSA peptide of the invention.

The invention includes cyclic derivatives of the PSA derived peptides of the invention. Cyclization allows the peptide to assume a more favourable conformation. Cyclization of the peptides may be achieved using techniques known in the art. In particular, disulphide bonds may be formed between two appropriately spaced components having free sulfhydryl groups. The bonds may be formed between side chains of amino acids, non-amino acid components or a combination of the two.

In a further aspect, the invention includes lipopeptide derivatives of the PSA derived peptides of the invention. Lipopeptides enhance the induction of CTL responses against antigens in vivo (See e.g. Deres et al., Nature 342, 561-564 (1989); Loing et al., J. Immune. 164(2), 900-907 (2000)) and constitute potent adjuvants in parenteral and mucosal immunization (Baier et al., Immunobiology 201, 391-405 (2000)). The lipopeptides of the present invention comprise a PSA derived peptide and one or more chains derived from fatty acids and/or steroid groups, and also include synthetic lipopeptides. The lipopeptides may be prepared using techniques known in the art. In particular, the fatty acids and/or steroid groups may be coupled on the alpha-NH₂ or epsilon-NH₂ functional groups of the amino acid residues of the PSA derived peptide.

Peptides of the present invention may be converted into pharmaceutical salts by reacting with inorganic acids including hydrochloric acid, sulphuric acid, hydrobromic acid, phosphoric acid, etc., or organic acids including formic acid, acetic acid, propionic acid, glycolic acid, lactic acid, pyruvic acid, oxalic acid, succinic acid, malic acid, tartaric acid, citric acid, benzoic acid, salicylic acid, benzenesulphonic acid, and toluenesulphonic acids.

The peptides of the invention may be prepared as N-terminal or C-terminal fusion proteins. The fusion proteins may be prepared by fusing, through recombinant techniques or by chemical crosslinking, the N terminal or C-terminal of the peptide, and the sequence of a selected protein or selectable marker with a desired biological function. The resultant fusion proteins contain the peptide fused to the selected protein or marker protein as described herein. Examples of proteins which may be used to prepare fusion proteins include immunoglobulins, glutathione-S-transferase, hemagglutinin, and truncated myc.

The peptides of the invention may be used to prepare monoclonal or polyclonal antibodies. Conventional methods can be used to prepare the antibodies. As to the details relating to the preparation of monoclonal antibodies reference can be made to Goding, J. W., Monoclonal Antibodies: Principles and Practice, 2nd Ed., Academic Press, London, 1986. As discussed below, the antibodies may be used to identify proteins similar or related to PSA.

The peptides and antibodies specific for the peptides of the invention may be labelled using conventional methods with various enzymes, fluorescent materials, luminescent materials and radioactive material. Suitable enzymes, fluorescent materials, luminescent materials, and radioactive material are well known to the skilled artisan. Labelled antibodies specific for the peptides of the invention may be used to screen for proteins similar or related to PSA as discussed in further detail below. Furthermore, the practice of the present invention will employ (unless otherwise indicated) conventional techniques of immunology, molecular biology, cell biology and recombinant DNA technology which are within the skill of the art.

II. Nucleic Acid Molecules of the Invention

The present invention also includes isolated nucleic acid molecules encoding the PSA derived peptides of the invention.

The term “isolated” refers to a nucleic acid substantially free of cellular material or culture medium when produced by recombinant DNA techniques, or chemical precursors, or other chemicals when chemically synthesized. The term “nucleic acid” is intended to include DNA and RNA and can be either double stranded or single stranded. As such, these nucleic acids comprise the relevant base sequences coding for the aforementioned PSA peptides. For purposes of definitiveness, the “relevant base sequences coding for the aforementioned PSA peptides” further encompasses complementary nucleic acid sequences.

In an embodiment of the invention, isolated nucleic acid molecules are provided having sequences which encode PSA peptides having the amino acid sequences as shown in SEQ ID NOS:1-3.

In a preferred embodiment, the invention provides isolated nucleic acid sequences comprising:

(a) nucleic acid sequences as shown in SEQ ID NOS:7-9 wherein T can also be U;

(b) nucleic acid sequences that are complementary to the nucleic acid sequences of (a);

(c) nucleic acid sequences that have substantial sequence homology to the nucleic acid sequences of (a) or (b);

(d) nucleic acid sequences that are analogs of the nucleic acid sequences of (a), (b) or (c); or

(e) nucleic acid sequences that hybridize to the nucleic acid sequences of (a), (b), (c) or (d) under stringent hybridization conditions.

The term “sequence that has substantial sequence homology” means those nucleic acid sequences which have slight or inconsequential sequence variations from the sequences in (a) or (b), i.e., the sequences function in substantially the same manner and can be used to elicit an immune response. The variations may be attributable to local mutations or structural modifications. Nucleic acid sequences having substantial homology include nucleic acid sequences having at least 65%, more preferably at least 85%, and most preferably 90-95% identity with the nucleic acid sequences as shown in SEQ ID NOS:7-9.

The term “sequence that hybridizes” means a nucleic acid sequence that can hybridize to a sequence of (a), (b), (c) or (d) under stringent hybridization conditions. Appropriate “stringent hybridization conditions” which promote DNA hybridization are known to those skilled in the art, or may be found in Current Protocols in Molecular Biology, John Wiley & Sons, N.Y. (1989), 6.3.1-6.3.6. For example, the following may be employed: 6.0×sodium chloride/sodium citrate (SSC) at about 45° C., followed by a wash of 2.0×SSC at 50° C.; 0.2×SSC at 50° C. to 65° C.; or 2.0×SSC at 44° C. to 50° C. The stringency may be selected based on the conditions used in the wash step. For example, the salt concentration in the wash step can be selected from a high stringency of about 0.2×SSC at 50° C. In addition, the temperature in the wash step can be at high stringency conditions, at about 65° C.

The term “a nucleic acid sequence which is an analog” means a nucleic acid sequence which has been modified as compared to the sequence of (a), (b) or (c) wherein the modification does not alter the utility of the sequence as described herein. The modified sequence or analog may have improved properties over the sequence shown in (a), (b) or (c). One example of a modification to prepare an analog is to replace one of the naturally occurring bases (i.e. adenine, guanine, cytosine or thymidine) of the sequences shown in SEQ ID NOS:7-9, with a modified base such as such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules shown in SEQ ID NOS:7-9. For example, the nucleic acid sequences may contain phosphorothioates, phosphotriesters, methyl phosphonates, and phosphorodithioates.

A further example of an analog of a nucleic acid molecule of the invention is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complimentary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequences.

It will be appreciated that the invention includes nucleic acid molecules encoding elongations of peptides of the invention, and analogs and homologs of peptides of the invention and elongations thereof, as described above.

Isolated and purified nucleic acid molecules having sequences which differ from the nucleic acid sequence of the invention due to degeneracy in the genetic code are also within the scope of the invention. Such nucleic acids encode functionally equivalent peptides but differ in sequence from the above mentioned sequences due to degeneracy in the genetic code.

An isolated nucleic acid molecule of the invention which comprises DNA can be isolated by preparing a labelled nucleic acid probe based on all or part of the nucleic acid sequences of the invention and using this labelled nucleic acid probe to screen an appropriate DNA library (e.g. a cDNA or genomic DNA library). For example, a genomic library isolated can be used to isolate a DNA encoding a novel protein of the invention by screening the library with the labelled probe using standard techniques. Nucleic acids isolated by screening of a cDNA or genomic DNA library can be sequenced by standard techniques.

An isolated nucleic acid molecule of the invention which is DNA can also be isolated by selectively amplifying a nucleic acid encoding a novel peptide of the invention using the polymerase chain reaction (PCR) methods and cDNA or genomic DNA. It is possible to design synthetic oligonucleotide primers from the nucleic acid sequence of the invention for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. It will be appreciated that cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA encoding a novel peptide of the invention into an appropriate vector which allows for transcription of the cDNA to produce an RNA molecule which encodes a peptide of the invention. For example, a cDNA can be cloned downstream of a bacteriophage promoter, (e.g., a T7 promoter) in a vector, cDNA can be transcribed in vitro with T7 polymerase, and the resultant RNA can be isolated by standard techniques.

A nucleic acid molecule of the invention may also be chemically synthesized using standard techniques. Various methods of chemically synthesizing polydeoxynucleotides are known, including solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a novel peptide of the invention may be accomplished by expressing the cDNA in an appropriate host cell by standard techniques, and testing the activity of the protein using the methods as described herein. A cDNA having the activity of a novel peptide of the invention so isolated can be sequenced by standard techniques, such as dideoxynucleotide chain termination or Maxam-Gilbert chemical sequencing, to determine the nucleic acid sequence and the predicted amino acid sequence of the encoded peptide.

The sequence of a nucleic acid molecule of the invention may be inverted relative to its normal presentation for transcription to produce an antisense nucleic acid molecule which are more fully described herein. Preferably, an antisense sequence is constructed by inverting a region preceding the initiation codon or an unconserved region. In particular, the nucleic acid sequences contained in the nucleic acid molecules of the invention or a fragment thereof, may be inverted relative to its normal presentation for transcription to produce antisense nucleic acid molecules.

The invention also provides nucleic acids encoding fusion proteins comprising a novel peptide of the invention and a selected protein, or a selectable marker protein (see below).

III. Preparation of the PSA Derived Peptides

The PSA peptides of the invention may be prepared using a variety of methods known to one skilled in the art. Accordingly, PSA peptides may be prepared by chemical synthesis using techniques well known in the chemistry of proteins such as solid phase synthesis (Merrifield, J. Am. Chem. Assoc. 85:2149-2154 (1964)) or synthesis in homogenous solution (Houbenweyl, Methods of Organic Chemistry, ed. E. Wansch, Vol. 15, pts. I and II, Thieme, Stuttgart (1987)).

The peptides of the invention may also be produced by recombinant DNA technology. To prepare the peptides of the invention by recombinant DNA techniques, a DNA sequence encoding the PSA derived peptide must be prepared. Consequently, the present invention also provides purified and isolated nucleic acids having a nucleotide sequence coding for PSA derived peptides. In one embodiment of the invention, the nucleic acid has a sequence encoding a PSA derived peptide comprising an amino acid sequence of the formula I wherein the substituents are described above. In other embodiments, the DNA sequence encoding a PSA derived peptide of the formula I comprises a nucleotide sequence of ATG TGG GTC CCG GTT GTC TTC CTC (SEQ ID NO:7), GTT CTG GTG CAC CCC CAG TGG GTC CTC (SEQ ID NO:8), or AAA CTT CAG TGT GTG GAC CTC CAT GTT (SEQ ID NO:9).

The present invention also provides an expression vector comprising a DNA molecule encoding a PSA derived peptide adapted for transfection or transformation of a host cell. The nucleic acid molecules of the present invention may be incorporated in a known manner into an appropriate expression vector which ensures expression of the protein. Possible expression vectors include but are not limited to cosmids, plasmids, or modified viruses (e.g. replication defective retroviruses, adenoviruses and adeno-associated viruses). The vector should be compatible with the host cell used. The expression vectors are “suitable for transformation of a host cell”, which means that the expression vectors contain a nucleic acid molecule of the invention and regulatory sequences selected on the basis of the host cells to be used for expression, which is operatively linked to the nucleic acid molecule. “Operatively linked” is intended to mean that the nucleic acid is linked to regulatory sequences in a manner which allows expression of the nucleic acid.

The invention therefore contemplates a recombinant expression vector containing a nucleic acid molecule of the invention, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. 1990). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the native A and B chains and/or its flanking regions.

The recombinant expression vectors of the invention may also contain a marker gene which facilitates the selection of host cells transformed or transfected with a recombinant molecule of the invention. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

Recombinant expression vectors can be introduced into host cells to produce a transformant host cell. The term “transformant host cell” is intended to include prokaryotic and eukaryotic cells which have been transformed or transfected with a recombinant expression vector of the invention. The terms “transformed with”, “transfected with”, “transformation” and “transfection” are intended to encompass introduction of nucleic acid (e.g. a vector) into a cell by one of many possible techniques known in the art. Prokaryotic cells can be transformed with nucleic acid by, for example, electroporation or calcium-chloride mediated transformation. Nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

More particularly, bacterial host cells suitable for carrying out the present invention include E. coli, B. subtilis, Salmonella typhimurium, and various species within the genus' Pseudomonas, Streptomyces, and Staphylococcus, as well as many other bacterial species well known to one of ordinary skill in the art. Suitable bacterial expression vectors preferably comprise a promoter which functions in the host cell, one or more selectable phenotypic markers, and a bacterial origin of replication. Representative promoters include the β-lactamase (penicillinase) and lactose promoter system (see Chang et al., Nature 275:615 (1978)), the trp promoter (Nichols and Yanofsky, Meth. in Enzymology 101:155, 1983) and the tac promoter (Russell et al., Gene 20: 231, 1982). Representative selectable markers include various antibiotic resistance markers such as the kanamycin or ampicillin resistance genes. Suitable expression vectors include but are not limited to bacteriophages such as lambda derivatives or plasmids such as pBR322 (see Bolivar et al., Gene 2:9S, (1977)), the pUC plasmids pUC18, pUC19, pUC118, pUC119 (see Messing, Meth in Enzymology 101:20-77, 1983 and Vieira and Messing, Gene 19:259-268 (1982)), and pNH8A, pNH16a, pNH18a, and Bluescript M13 (Stratagene, La Jolla, Calif.). Typical fusion expression vectors which may be used are discussed above, e.g. pGEX (Amrad Corp., Melbourne, Australia), pMAL (New England Biolabs, Beverly, Mass.) and pRIT5 (Pharmacia, Piscataway, N.J.). Examples of inducible non-fusion expression vectors include pTrc (Amann et al., Gene 69:301-315 (1988)) and pET 11d (Studier et al., Gene Expression Technology: Methods in Enzymology 185, Academic Press, San Diego, Calif., 60-89 (1990)).

Yeast and fungi host cells suitable for carrying out the present invention include, but are not limited to Saccharomyces cerevisiae, Schizosaccharomyces pombe, the genera Pichia or Kluyveromyces and various species of the genus Aspergillus. Examples of vectors for expression in yeast S. cerivisiae include pYepSec1 (Baldari. et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art.(see Hinnen et al., Proc. Natl. Acad. Sci. USA 75:1929 (1978); Itoh et al., J. Bacteriology 153:163 (1983), and Cullen et al. (Bio/Technology 5:369 (1987)).

Mammalian cells suitable for carrying out the present invention include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the proteins of the invention may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, pAS2022, pAS2023, and pAS2034).

Insect cells suitable for carrying out the present invention include cells and cell lines from Bombyx or Spodotera species. Baculovirus vectors available for expression of proteins in cultured insect cells (SF 9 cells) include the pAc series (Smith et al., Mol. Cell Biol. 3:2156-2165 (1983)) and the pVL series (Lucklow, V. A., and Summers, M. D., Virology 170:31-39 (1989)). Some baculovirus-insect cell expression systems suitable for expression of the recombinant proteins of the invention are described in PCT/US/02442.

The recombinant expression vectors containing the nucleotide sequences encoding the PSA derived peptides may also contain genes which encode a fusion moiety (i.e. a “fusion protein”) which provides increased expression of the recombinant peptide; increased solubility of the recombinant peptide; and aid in the purification of the target recombinant peptide by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein. By way of illustration, the DNA sequence encoding an enhanced affinity PSA derived peptide may be cloned into a pGEX-type plasmid for co-expression with a 26 kD protein glutathione-S-transferase (GST): pGEX-2T, pGEX-2TK, pGEX-3X, pGEX-1T, pGEX-4T, pGEX-5X. Said plasmids are transformed into Escherichia coli HB101 cells, and positive clones can then be selected by standard hybridization techniques identifying GST-PSA peptide fusion protein. Colonies with the pGEX-2T plasmids containing DNA encoding an enhanced affinity PSA peptide may be grown in 5 mL of LB/amp medium at 37° C. for overnight. Isopropylthio—D-galactoside is added to the culture to a final concentration of 0.1 mM to induce the biosynthesis of the fusion protein. The cultures are grown for 90 min post induction and the cells are harvested by centrifugation and lysed in 1 mL of 50 mM Tris-HCl, pH 8.0, containing 2 M urea and 1% Triton X-100. Cells can then be further disrupted by sonication and centrifuged at 15,000×g for 20 min to separate soluble from insoluble fractions. The supernatant containing the GST-PSA peptide fusion protein is then used for extraction of the fusion protein by standard procedures (GST Purification Module: Pharmacia Cat. #27-4570-01, -02). Subsequently, the fusion protein may be treated with an enzyme to release the enhanced affinity PSA peptide (thrombin for pGEX-2T, pGEX-2TK, PGEX-1T, pGEX-4T; factor Xa for pGEX-3X and pGEX-5X).

Additional embodiments of the invention encompass recombinant nucleic acids further comprising inserts. These inserts code for the PSA peptides hereinbefore described. Further embodiments encompass recombinant nucleic acids wherein the insert comprises a sequence chosen from the group consisting of SEQ ID NO:7, SEQ ID NO:8, and SEQ ID NO:9. As defined herein, “recombinant nucleic acids” encompass (but is not limited to) viruses, bacterial DNA, naked/free DNA and RNA.

IV. Applications of the Peptides and Nucleic Acids

The inventors have demonstrated that the PSA derived peptides of the present invention are immunogenic and capable of eliciting immune responses in vivo. Consequently, the present invention includes the use of one or more PSA derived peptides of the invention to modulate immune responses. Accordingly, the present invention provides a method of modulating immune responses comprising administering an effective amount of a PSA derived peptide or a nucleic acid molecule encoding a PSA peptide of the invention to a cell or animal in need thereof.

The term “animal” as used herein includes all members of the animal kingdom including mammals, preferably humans.

The term “eliciting an immune response” is defined as initiating, triggering, causing, enhancing, improving or augmenting any response of the immune system, for example, of either a humoral or cell-mediated nature. The initiation or enhancement of an immune response can be assessed using assays known to those skilled in the art including, but not limited to, antibody assays (for example ELISA assays), antigen specific cytotoxicity assays and the production of cytokines (for example ELISPOT assays). Preferably, the peptides and nucleic acids of the present invention, and the method of the present invention trigger or enhance a cellular immune response, more preferably a cytotoxic T cell response.

The term “effective amount” as used herein means an amount effective, at dosages and for periods of time necessary to achieve desired results.

More specifically, the peptides of the invention may be used in the prophylaxis or treatment of pathological conditions such as cancer, including tumor metastasis, in a mammal.

Additional embodiments of the invention encompass compositions comprising PSA peptides, and/or nucleic acids coding for PSA peptides, and/or recombinant nucleic acids into which has been inserted a nucleic acid sequence coding for PSA peptide(s) (all of which have been herein before described). The peptides and nucleic acid molecules may be formulated into pharmaceutical compositions for administration to subjects in a biologically compatible form suitable for administration. By biologically compatible form suitable for administration is meant a form of the substance to be administered in which any toxic effects are outweighed by the therapeutic effects. The substances may be administered to living organisms including humans, and animals in a therapeutically effective amount. Administration of an effective amount of the pharmaceutical compositions of the present invention is defined as an amount effective, at dosages and for periods of time necessary to achieve the desired result. For example, a therapeutically active amount of a substance may vary according to factors such as the disease state, age, sex and weight of the individual, and the ability of peptide to elicit a desired response in the individual. Dosage regime may be adjusted to provide the optimum therapeutic response. For example, several divided doses may be administered daily or the dose may be proportionally reduced as indicated by the exigencies of the therapeutic situation.

The active substance may be administered in a convenient manner such as by topical or transdermal application, injection (subcutaneous, intravenous, etc.), oral administration, inhalation, or rectal administration. Depending on the route of administration, the active substance may be coated in a material to protect the compound from the action of enzymes, acids and other natural conditions which may inactivate the compound.

Several modes of administration are available when using a composition containing a nucleic acid molecule encoding a PSA derived peptide of the invention. Recombinant molecules comprising a nucleic acid sequence encoding a PSA derived protein (as described above), or fragment thereof, may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes. Recombinant molecules may also be delivered in the form of an aerosol or by lavage. The nucleic acid molecules of the invention may also be applied extracellularly such as by direct injection into cells.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences (1985), Mack Publishing Company, Easton, Pa., USA). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456.

Compositions for injection include, albeit not exclusively, the peptides or nucleic acids in association with one or more pharmaceutically acceptable vehicles or diluents, and contained in buffered solutions with a suitable pH and iso-osmotic with the physiological fluids. Any pharmaceutically suitable diluent can be used in the composition for injections: distilled water, physiological or a salt solution, and/or a buffer solution. The composition for injections may be prepared by conventional volume-weight procedures. A certain amount of the peptide is diluted to the necessary volume with a diluent or solvent. The solution is then filtered through sterilized filters, bottled or ampouled. The resultant solution is a stable transparent liquid, and does not contain any chemical or other impurities.

Solid form preparations for oral administration can be made in the form of tablets, powders, or capsules. It may contain a medium for the active substance and other additives, including dyes, aromas, etc.

The compositions and treatments are indicated as therapeutic agents or treatments either alone or in conjunction with other therapeutic agents or other forms of treatment.

Immunogenicity can be significantly improved if the immunizing agent(s) (i.e. PSA peptide, and/or nucleic acid coding therefor, recombinant nucleic acids) and/or composition is, regardless of administration format, co-immunized with an adjuvant. Commonly, adjuvants are used as an 0.05 to 1.0 percent solution in phosphate-buffered saline. Adjuvants enhance the immunogenicity of an immunogen but are not necessarily immunogenic themselves. Adjuvants may act by retaining the immunogen locally near the site of administration to produce a depot effect facilitating a slow, sustained release of immunogen to cells of the immune system. Adjuvants can also attract cells of the immune system to an immunogen depot and stimulate such cells to elicit immune responses. As such, embodiments of this invention encompass compositions further comprising adjuvants.

Adjuvants have been used for many years to improve the host immune responses to, for example, vaccines. Intrinsic adjuvants (such as lipopolysaccharides) normally are the components of killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Some of these adjuvants are toxic, however, and can cause undesirable side-effects making them unsuitable for use in humans and many animals. Indeed, only aluminum hydroxide and aluminum phosphate (collectively commonly referred to as alum) are routinely used as adjuvants in human and veterinary vaccines. The efficacy of alum in increasing antibody responses to diphtheria and tetanus toxoids is well established. Notwithstanding, it does have limitations. For example, alum is ineffective for influenza vaccination and inconsistently elicits a cell mediated immune response with other immunogens. The antibodies elicited by alum-adjuvanted antigens are mainly of the IgG1 isotype in the mouse, which may not be optimal for protection by some vaccinal agents.

A wide range of extrinsic adjuvants can provoke potent immune responses to immunogens. These include saponins complexed to membrane protein antigens (immune stimulating complexes), pluronic polymers with mineral oil, killed mycobacteria and mineral oil, Freund's complete adjuvant, bacterial products such as muramyl dipeptide (MDP) and lipopolysaccharide (LPS), as well as lipid A, and liposomes.

In one aspect of this invention, adjuvants useful in any of the embodiments of the invention described herein are as follows. Adjuvants for parenteral immunization include aluminum compounds (such as aluminum hydroxide, aluminum phosphate, and aluminum hydroxy phosphate). The antigen can be precipitated with, or adsorbed onto, the aluminum compound according to standard protocols. Other adjuvants such as RIBI (ImmunoChem, Hamilton, Mont.) can also be used in parenteral administration.

Adjuvants for mucosal immunization include bacterial toxins (e.g., the cholera toxin (CT), the E. coli heat-labile toxin (LT), the Clostridium difficile toxin A and the pertussis toxin (PT), or combinations, subunits, toxoids, or mutants thereof. For example, a purified preparation of native cholera toxin subunit B (CTB) can be of use. Fragments, homologs, derivatives, and fusion to any of these toxins are also suitable, provided that they retain adjuvant activity. Preferably, a mutant having reduced toxicity is used. Suitable mutants have been described (e.g., in WO 95/17211 (Arg-7-Lys CT mutant), WO 96/6627 (Arg-192-Gly LT mutant), and WO 95/34323 (Arg-9-Lys and Glu-129-Gly PT mutant)). Additional LT mutants that can be used in the methods and compositions of the invention include, for example Ser-63-Lys, Ala-69-Gly, Glu-110-Asp, and Glu-112-Asp mutants. Other adjuvants (such as a bacterial monophosphoryl lipid A (MPLA) of various sources (e.g., E. coli, Salmonella minnesota, Salmonella typhimurium, or Shigella flexneri, saponins, or polylactide glycolide (PLGA) microspheres) can also be used in mucosal administration.

Adjuvants useful for both mucosal and parenteral immunization include polyphosphazene (for example, WO 95/2415), DC-chol (3 b-(N-(N′,N′-dimethyl aminomethane)-carbamoyl) cholesterol (for example, U.S. Pat. No. 5,283,185 and WO 96/14831) and QS-21 (for example, WO 88/9336).

An animal may be immunized with a PSA peptide(s), a nucleic acid coding therefor, a recombinant nucleic acid wherein a nucleic acid coding for a PSA peptide is inserted therein, and/or a composition of this invention by any conventional route as is known to one skilled in the art. This may include, for example, immunization via a mucosal (e.g., ocular, intranasal, oral, gastric, pulmonary, intestinal, rectal, vaginal, or urinary tract) surface, via the parenteral (e.g., subcutaneous, intradermal, intramuscular, intravenous, or intraperitoneal) route or intranodally. Preferred routes depend upon the choice of the immunogen as will be apparent to one skilled in the art. The administration can be achieved in a single dose or repeated at intervals. The appropriate dosage depends on various parameters understood by skilled artisans such as the immunogen itself (i.e. peptide vs. nucleic acid (and more specifically type thereof), the route of administration and the condition of the animal to be vaccinated (weight, age and the like).

Accordingly, embodiments of this invention encompass methods of eliciting immune responses in animals comprising administering effective amounts of PSA peptide(s), and/or nucleic acid(s) coding therefore, and/or recombinant nucleic acid(s) wherein a nucleic acid coding for a PSA peptide is inserted therein, and/or compositions of the invention. The present invention also includes methods of treating cancer comprising administering effective amounts of PSA peptide(s), and/or nucleic acid(s) coding therefore, and/or recombinant nucleic acid(s) wherein a nucleic acid coding for a PSA peptide is inserted therein, and/or compositions of the invention. In a preferred embodiment, the methods of the invention are utilized to treat prostate cancer.

A further embodiment of this invention encompasses a use of effective amounts of PSA peptide(s), and/or nucleic acid(s) coding therefore, and/or recombinant nucleic acid(s) wherein a nucleic acid coding for a PSA peptide is inserted therein, and/or a composition of the present invention to elicit an immune response in an animal preferably to treat cancer, more preferably prostate cancer. The present invention further includes a use of effective amounts of PSA peptide(s), and/or nucleic acid(s) coding therefore, and/or recombinant nucleic acid(s) wherein a nucleic acid coding for a PSA peptide is inserted therein, and/or a composition of the present invention to prepare a medicament to elicit an immune response in animal, preferably to treat cancer, more preferably prostate cancer.

The following non-limiting examples are illustrative of the present invention:

### EXAMPLES

Experimental

The analysis of peptides isolated from human class 1 MHC complexes have revealed that peptides which are favored to bind and lodge into the peptide-binding groove of the human MHC class 1 molecule (such as HLA-A0201) are typically 9 amino acids long (however, peptides of 8-13 amino acids have also been observed). In the majority of cases, these nonamers contain two anchor residues; the first proximal to the amino-(N—) terminus, and the second associated with the carboxy-(C—) terminus. These anchor residues interact with the respective ‘pockets’ of the peptide-binding groove of the MHC molecules. The amino-(N—) associated anchor residue is typically leucine (L) or methionine (M); the carboxy-(C—) associated residue is typically leucine (L) or valine (V).

Methods of peptide synthesis, cell culture propagation and assays of cytotoxicity are amply reported in the scientific literature and are well within the scope of those skilled in the art; as such, detailed particulars of these methods will not be explicitly discussed.

### Example 1

A number of PSA derived peptide sequences were identified that correspond to the above-mentioned Formula I. These are outlined in Table 1.

Peptide Synthesis

Solid phase peptide syntheses were conducted on an ABI 430A automated peptide synthesizer according to the manufacturer's standard protocols. The peptides were cleaved from the solid support by treatment with liquid hydrogen fluoride in the presence of thiocresole, anisole, and methyl sulfide. The crude products were extracted with trifluoroacetic acid (TFA) and precipitated with diethyl ether. All peptides were stored in lyophilized form at −20° C.

| | | |
|---|---|---|
| CLP313 | MWVPVVFL | (SEQ ID NO:1) |
| CLP314 | VLVHPQWVL | (SEQ ID NO:2) |
| CLP316 | KLQCVDLHV | (SEQ ID NO:3) |
| CLP315 | DLPTQEPAL | (SEQ ID NO:4) |
| CLP317 | DLHVISNDV | (SEQ ID NO:5) |
| CLP320 | PLYDMSLLK | (SEQ ID NO:6) |

Prior to immunization of animals, peptides were dissolved in 100% Dimethylsulphoxide (DMSO), subsequently diluted with sterile distilled water (DW) to a final DMSO concentration 5.0%, and stored at −20° C. pending use.

### Example 2

Nucleic Acid Sequences Coding for PSA Derived Peptides

The nucleic acid sequence coding for the identified PSA peptides (i.e. SEQ ID. NOs:1-6) were deduced by reference to the nucleic acid sequence disclosed in the publications of Henttu and Vihko ((1989) Biochem and Biophys Res Commun 160:903-910), Lundwall and Lilja ((1987) FEBS Lett. 214:317-322) and Schulz et al. ((1988) Nucleic Acids Research 16:6226).

The coding strand nucleic acid sequences were:

| Peptide | Nucleic Acid Sequence | |
|---|---|---|
| CLP313(SEQ ID NO:1) | ATGTGGGTCCCGGTTGTCTTCCTC | (SEQ ID NO:7) |
| CLP314(SEQ ID NO:2) | GTTCTGGTGCACCCCCAGTGGGTCCTC | (SEQ ID NO:8) |
| CLP316(SEQ ID NO:3) | AAACTTCAGTGTGTGGACCTCCATGTT | (SEQ ID NO:9) |
| CLP315(SEQ ID NO:4) | GACCTGCCCACCCAGGAGCCAGCACTG | (SEQ ID NO:10) |
| CLP317(SEQ ID NO:5) | GACCTCCATGTTATTTCCAATGACGTG | (SEQ ID NO:11) |
| CLP320(SEQ ID NO:6) | CCGCTCTACGATATGAGCCTCCTGAAG | (SEQ ID NO:12) |

### Example 3

HLA-A0201 Binding of PSA Derived Peptides

The ability of the PSA derived peptides described in Table 1 to stabilize membrane-bound HLA-A0201 molecule was assessed utilizing the T2 cell line (Dr. Peter Creswell, Yale University). The cell line has been well documented to have a defective TAP (i.e. Transporter for Antigen Processing) transporter function. As a result, the majority of intracellularly generated peptides are not transported into the endoplasmic reticulum and thus are unable to associate with newly synthesized HLA class 1 MHC molecules (i.e. HLA-A0201; Salter, R D and Creswell, P. (1986) EMBO J 5:943). The majority of the HLA-A0201 molecules displayed on the surface of T2 cells are therefore empty (contain no peptides) and unstable. The stability of the surface HLA-A0201 molecules can be restored upon interaction with suitable exogenous peptides. The stabilization of the conformation of the class 1 MHC molecules is accompanied by the formation of an immunodominant epitope recognized by a mouse monoclonal antibody (designated BB7.2; American Type Culture Collection (ATCC)). Thus, the detection of this specific epitope is indicative of stable membrane-bound HLA-A0201 molecules loaded with peptide. Subsequent dissociation of peptides from the HLA class 1 MHC molecules results in the loss of BB7.2 monoclonal antibody binding.

T2 cells were propagated in Iscove's complete medium (iscove's medium supplemented with 10% heat-inactivated bovine serum, 120.0 units per ml of penicillin G sodium, 120 μg per ml of streptomycin sulphate, and 0.35 mg per ml of L-glutamine). The ability of PSA peptides (Table 1) to bind and stabilize surface HLA-A0201 molecules on T2 cells was determined utilizing a protocol documented in the art (Deng, Y. (1997) J Immune 158:1507-1515). In essence, 0.5×10⁶ T2 cells were incubated with 125.0 μM of the test peptide in 200.0 μl of serum-free culture medium (Iscove's medium supplemented with 120.0 units per ml of penicillin G sodium, 120.0 μg per ml of streptomycin sulphate and 0.35 mg per ml of L-glutamine) overnight at 37° C. Subsequently, cells were washed twice with Iscove's medium (without bovine serum) to remove free PSA peptide. To the cell pellet was added 1.0 ml of complete Iscove's medium (supplemented with fetal bovine serum (10% final)) containing 5.0 μg per ml of brefeldin A (Sigma), 12.5 μg per ml of anisomycin (Sigma) and 5.0 μg per ml of cyclohexamide (Sigma). The samples were incubated for 3.0 hr in a 37° C. CO₂ incubator. The cells were subsequently incubated 30 min on ice prior to two washes with ice-cold PBA (phosphate buffered saline (pH 7.2) containing 2.0% bovine serum). 100.0 μl of PBA containing 5.0 μg of monoclonal antibody BB7.2 was added to each test sample. The reaction was allowed to proceed on ice for 45 min. Cells were then washed three times with ice-cold PBA. The binding of BB7.2 was detected via the addition of 100.0 μl of PBA containing 1.0 pg of goat anti-mouse IgG-Fc fluorescein (FITC) conjugate (BETHYL Laboratories Inc.). After a 30 min incubation on ice, cells were twice washed with ice-cold PBA, and twice with ice-cold PBS (pH 7.2). Cells were immediately fixed by the addition of 50.0 μl of 1.0% paraformaldehyde. Samples were analyzed by Flow Cytometry. Results were expressed in units of Fluorescence Index (FI), calculated by the equation:

Mean Fluorescence (MF) of experimental sample (peptide treated)−MF of control sample (cells not peptide treated) MF of control sample (cells not peptide treated)

An FI value of 0.5 or greater was deemed to be significant.

The results depicted in Table 1 reveal that a negative control peptide (CLP-320) containing a binding motif for the gene product of HLA-A1 does not exhibit binding to the membrane-bound HLA-A0201 molecules on T2 cells. Of the 6 PSA peptides tested, 3 (i.e. CLP-313, CPL-314, CPL-316) demonstrated HLA-A0201 binding as judged by Fluorescence Index (FI) values (Table 1).

### Example 4

Immunogenicity of PSA Derived Peptides

The A2Kb transgenic mouse was used to assess the immunogenicity of the HLA-A0201 binding PSA peptides. Mice of the B1O background (transgenic for the A2Kb chimeric gene) were purchased from the Scripps Clinic in California, USA. Mice were injected subcutaneously at the base of the tail with a dose of inoculum prepared by emulsifying 100.0 μg of the test peptide and 100.0 μg of an I-A^{b}-restricted peptide (described in Milich, D. R. et al. (1987) J. Immune 139: 1223-1231) in incomplete Freund's adjuvant (IFA). Spleens of the experimental animals were collected on the 10th or 11th day post immunization. Spleenocytes of the experimental mice were prepared and cultured to enrich for CTLs before being assessed for effector activity. In vitro re-stimulation of the in vivo generated CTLs was performed by co-culturing in a 25 cm² tissue culture flask 3×10⁷ responder cells (i.e. spleenocytes) with 1.3×10⁷ irradiated autologous LPS (lipopolysaccharide)-blasts which had been pulsed with the respective peptide (100.0 μg per 10⁸ cells). Cultures were kept in a 37° C., humidified CO₂ incubator for 6-7 days before being tested for effector function in a standard 4 hr in vitro ⁵¹Cr-release CTL assay as follows. The responders were harvested from the 6-7 day cultures and washed twice with RPMI-1640 medium (without bovine serum). The positive target was created by incubating 3-5×10⁶ P815-A2Kb transfectant with 100.0 μg of the specified peptide overnight in a 26° C. water bath. The target cells were then labeled with ⁵¹Cr at 250.0 μCi per 1×10⁶ cells for 1 hr in the presence of 15.0 μg of the same test peptides. After washing twice with complete medium to remove excess free ⁵¹Cr, the targets were incubated at 2.5×10³ with different numbers of the responders for 4 hr in a 37° C. CO₂ incubator. Supernatant aliquots were then removed and counted for radioactivity.

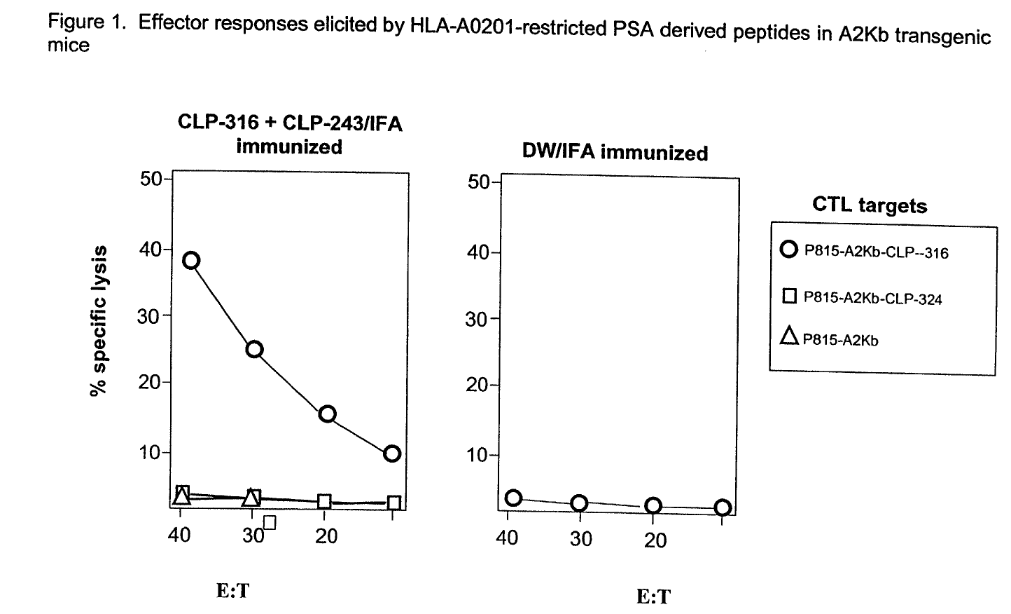

CLP-316 was selected as a representative PSA peptide for this study. The results depicted in FIG. 1 reveal that CLP-316 was immunogenic and capable of eliciting an epitope-specific CTL response.

Whereas the invention is susceptible to various modifications and/or alternate forms, specific embodiments have been shown by way of example and are herein described in detail. However, it should be understood that it is not intended to limit the invention to the particular embodiments shown, but on the contrary, the invention is to cover all modifications, equivalents, and/or alternatives following within the spirit and scope of the invention as defined by the appended claims.

All publications, patents and patent applications referred to herein, are herein incorporated by reference in their entirety to the same extent as if each individual publication, patent or patent application was specifically and individually indicated to be incorporated by reference in its entirety.

TABLE 1
Capacity of PSA derived peptides to bind and
stabilize HLA-A0201 molecules on T2 cells

| | | | | Fluorescence |
|---|---|---|---|---|
| Peptide | Amino Acid Position (Sequence)* | | SEQ ID | Index (FI) |
| CLP-313 | 1-8 | (MWVPWFL) | 1 | 1.0 |
| CLP-314 | 53-61 | (VLVHPQWVL) | 2 | 1.1 |
| CLP-316 | 170-178 | (KLQCVDLHV) | 3 | 2.7 |
| CLP-315 | 140-148 | (DLPTQEPAL) | 4 | 0 |
| CLP-317 | 175-183 | (DLHVISNDV) | 5 | 0 |
| CLP-320 | 99-107 | (PLYDMSLLK)*** | 6 | 0.2** |

*Amino acid position pertains to that of the preproprotein.
**Analysis did not include 3 hr de-stabilization step
***Negative Control

We claim:

1. A prostate-specific antigen (PSA) derived peptide that is capable of eliciting an immune response comprising a sequence of the Formula I:
Xₙ—X₁—X—X—X—X—X—X—X₂
wherein
n=0 or 1;
each X₁ is independently selected from leucine or methionine;
each X₂ is independently selected from valine or leucine; and
each X is independently selected from any amino acid,
and fragments, elongations, analogs or derivatives of the PSA derived peptide.

2. A PSA derived peptide according to claim 1 selected from the group consisting of MWVPVVFL (SEQ ID NO: 1), VLVHPQWVL (SEQ ID NO: 2), and KLQCVDLHV (SEQ ID NO: 3), or a fragment, analog, derivative or elongation of the PSA derived peptide.

3. A PSA derived peptide according to claim 1 selected from the group consisting of MWVVVFL (SEQ ID NO: 1), VLVHPQWVL (SEQ ID NO: 2), and KLQCVDLHV (SEQ ID NO: 3).

4. A fusion protein comprising the PSA peptide as described in claim 1.

5. A nucleic acid molecule encoding a PSA derived peptide according to claim 1.

6. A nucleic acid molecule encoding a PSA derived peptide according to claim 5 comprising:
(a) a nucleic acid sequence as shown in any one of SEQ ID NOS:7-9 wherein T can also be U;
(b) a nucleic acid sequence that is complementary to a nucleic acid sequence of (a);
(c) a nucleic acid sequence that has substantial sequence homology to a nucleic acid sequence of (a) or (b);
(d) a nucleic acid sequence that is an analog of a nucleic acid sequence of (a), (b) or (c); or
(e) a nucleic acid sequence that hybridizes to a nucleic acid sequence of (a), (b), (c) or (d) under stringent hybridization conditions.

7. A nucleic acid molecule encoding a PSA derived peptide according to claim 5 having a sequence selected from the group consisting of: SEQ ID NO:7; SEQ ID NO:8; and SEQ ID NO:9.

8. An expression vector comprising a nucleic acid molecule of claim 5 and regulatory sequences suitable for expression of the nucleic acid molecule.

9. A host cell transformed with an expression vector of claim 8.

10. A composition for eliciting an immune response in an animal comprising an effective amount of a peptide according to claim 1 in admixture with a suitable diluent or carrier.

11. The composition of claim 10 further comprising an adjuvant.

12. A composition for eliciting an immune response in an animal comprising an effective amount of a nucleic acid according to claim 5 in admixture with a suitable diluent or carrier.

13. The composition of claim 12 further comprising an adjuvant.

14. A method of eliciting an immune response in an animal comprising administering an effective amount of a peptide according to claim 1 to the animal.

15. A method of eliciting an immune response in an animal comprising administering an effective amount of a fusion protein according to claim 4 to the animal.

16. A method of eliciting an immune response in an animal comprising administering an effective amount of a nucleic acid molecule according to claim 5 to the animal.

17. A method of eliciting an immune response in an animal comprising administering an effective amount of a composition according to claim 10 to the animal.

18. A method of treating cancer comprising administering to an animal an effective amount of a peptide according to claim 1.

19. A method of treating cancer comprising administering to an animal an effective amount of a fusion protein according to claim 4.

20. A method of treating cancer comprising administering to an animal an effective amount of a nucleic acid molecule according to claim 5.

21. A method of treating cancer comprising administering to an animal an effective amount of a composition according to claim 10.

22. A method according to claim 18 wherein the cancer is prostate cancer.
